(19) United States
(12) Patent Application Publication
(10) Pub. No.: US 2004/0115187 A1
(43) Pub. Date: Jun. 17, 2004

(54) Compositions comprising at least one glyconsidase, said compositions containing no proteases

(75) Inventors: Bruno Mehul, Villejuif (FR); Dominique Bernard, Paris (FR)

Correspondence Address: BURNS, DOANE, SWECKER & MATHIS, L.L.P., P.O. Box 1404, Alexandria, VA, 22313-1404, US

(73) Assignee: SOCIETE L'OREAL S.A., Paris (FR)

(21) Appl. No.: 10/436,159

(22) Filed: May 13, 2003

Related U.S. Application Data
(63) Continuation of application No. PCT/FR01/03551, filed on Nov. 13, 2001.

(30) Foreign Application Priority Data
Nov. 13, 2000 (FR) 00/14556

(51) Int. Cl. A61K 7/06; A61K 7/11; A61K 38/47

(52) U.S. Cl. 424/94.61; 424/70.14

(57) ABSTRACT

The present invention concerns compositions comprising at least a glycosidase but not a protease to promote scaling. The invention further concerns the use of the compositions for treating dry skin or for preventing or treating skin aging.

### CROSS-REFERENCE TO PRIORITY/PCT APPLICATIONS

This application claims priority under 35 U.S.C. § 119 of FR-00/14556, filed Nov. 13, 2000, and is a continuation of PCT/FR01/03551, filed Nov. 13, 2001 and designating the United States (published in the French language on May 16, 2002 as WO 02/038122 A3; the title and abstract were also published in English), both hereby expressly incorporated by reference.

The present invention relates to compositions that encourage desquamation. In particular, it relates to compositions that are free from proteases and comprising at least one glycosidase and/or a glycosidase activator and to their use in combating skin aging or dry skin.

Desquamation is a natural phenomenon linked to the fact that the epidermis, which constitutes the outer layer of the skin, is constantly regenerating itself. The epidermis is constituted by several strata of cells, the lowest of which, the stratum basale, is constituted by undifferentiated cells. Over time, those cells differentiate and migrate towards the surface of the epidermis to constitute the different strata thereof until they form corneocytes on the surface of the epidermis, i.e., the outermost layer, also known as the stratum corneum, which are removed by desquamation. That surface loss is compensated for by migration of cells from the stratum basale towards the surface of the epidermis. Thus, the skin is constantly being renewed.

Young skin renews its superficial layers every two to three weeks, while a mature skin can take twice as long. One consequence of this slowing down of epidermal renewal is the impression of dry skin. The longer the renewal process, the more reduced the natural moisturization of the skin surface.

Further, the slower the desquamation process, the more matte the skin becomes, and it loses its glow and freshness.

Forced removal of the stratum corneum accelerates epidermal renewal and can combat cutaneous aging; the skin regains a youthful fresh appearance and moisturization is improved. The skin loses its matte appearance and the complexion improves.

Cutaneous aging resulting from the effects of intrinsic or extrinsic factors on the skin result in the appearance of fine lines and wrinkles and yellowing of the skin, which develops a parchment-like appearance, possibly accompanied by the appearance of skin blemishes, by disorganization of elastin and collagen fibres which causes a loss of elasticity, suppleness and firmness and by the appearance of telangiectasias.

More particularly, certain of those signs of aging are linked to intrinsic or physiological aging, i.e., “normal” aging linked to age or chronobiology, while others are more specific to extrinsic aging, i.e., aging generally caused by the environment; more particularly, it is due to photo-aging due to exposure to the sun, to light or to any other radiation.

The invention is applicable to intrinsic or physiological aging and to extrinsic aging.

Skin changes due to intrinsic aging are the consequence of programmed genetic senescence involving endogenic factors. Intrinsic aging causes skin cell renewal to slow down, which essentially results in the appearance of clinical alterations such as a reduction in subcutaneous adipose tissue and the appearance of fine lines, and by histopathological changes such as an increase in the number and thickness of elastic fibres, a loss of vertical fibres in the elastic tissue membrane, and the presence of large irregular fibroblasts in the cells of this elastic tissue.

In contrast, extrinsic aging causes clinical alterations such as deep wrinkles and the formation of a soft and/or hide-like skin, and histopathological changes such as an excessive accumulation of elastic material in the upper dermis and degeneration of collagen fibres.

Proteolytic degradations observed during the desquamation process can be induced by proteases synthesized by the stratum corneum, in particular the stratum corneum chymotrypsin enzyme (SCCE). It has been shown that glycosidases of lysosomial origin are externalized during terminal differentiation of the epidermis (Nemanic et al, J of Invest Dermat, 1983, 81:28-33). However, characterization of substrates for said glycosidases in the stratum corneum has not advanced very far and the biological role of said glycosidases has not been clarified.

It has been observed that the presence of saccharide residues on the proteins protects desmosomal structures and the glycoproteins of the corneosomes from proteolytic degradation artificially induced by the addition of proteases (Walsh and Chapman (1990), Arch Dermatol Res 282: 304-310).

This observation leads to the hypothesis that endogenous glycosidases could encourage the action of proteases in the stratum corneum and accelerate desquamation.

It is also possible that the sugars generated by endogenous glycosidase activity could cause a direct cellular response, resulting in the release of corneocytes.

The development of cosmetics that encourage desquamation on applying them to the skin can thus combat dry skin and cutaneous aging in particular. A number of examples of cosmetic compositions encouraging desquamation have been described in the prior art. In particular, we can cite compositions containing retinoic acid, such as those described in United States patent U.S. Pat. No. 4,603,146, compositions containing α- or β-hydroxides (see for example, European application EP-A-0 413 528 or International application number WO-A-93/10756). However, those compositions cause side effects consisting of tingling, itching, burning and redness that are disagreeable for the user. Examples of compositions essentially comprising proteases or a combination of proteases and glycosidases have been described in the prior art (PCT WO-A-93/19732, Unilever plc; PCT WO-A-95/07687, Unilever plc). However, there are toxicological risks associated with the use of proteases in topical compositions and they can cause allergic reactions and irritation.

The invention is the result of studies carried out on the effect of exogenic glycosidases on desquamation. These studies have been carried out with the help of the development of an in vitro desquamation test suitable for molecule screening. Said studies have demonstrated that, surprisingly, an increase in glycosidase activity in the stratum corneum by adding exogenous glycosidases, if necessary in combination with a product that can stimulate endogenous glycosidase activity, is sufficient to encourage desquamation in the absence of an addition of exogenic proteases.

Consequently, the invention concerns compositions comprising at least one glycosidase encouraging desquamation as the active principle, said composition containing no proteases. It also concerns a composition comprising at least one glycosidase encouraging desquamation and a product that can stimulate the activity of said glycosidases.

The glycosidases included in the compositions of the invention are all enzymes that can have proteoglycanes, glycolipids and in general, glycoconjugated compounds of the stratum corneum as a substrate. Said enzymes are sialidases such as neuraminidases, mannosidases, galactosidases, glucosidases, N-acetyl-glucosaminidases, N-acetyl-galactosaminidases, chondroitinases, glucuronidases or hyaluronidases.

They can also be any glycosidase encouraging desquamation, selected by any glycosidase desquamation efficacy test (Lundström A and Egelrud T, Arch Dermatol Res (1990), 282: 234-237) or by a test comprising the following steps:

a) recovering samples of stratum corneum from an individual;

b) fixing the sample onto a support;

c) bringing the sample fixed on the support into contact with a buffered solution containing the glycosidase to be tested;

d) recovering insoluble material released into the buffered solution;

e) assaying the keratins present in the insoluble material and quantifying them with respect to a test control without glycosidase.

In the present test, the keratins assayed in step e) in the insoluble material reflect the quantity of corneocytes released. It can thus test the “pro-desquamatizing” effect of a product, more particularly of glycosidases.

The invention pertains to compositions comprising one or more glycosidases as the active principle, said compositions containing no proteases.

The glycosidases of the invention also encompass cellulases for which no endogenous substrate in the stratum corneum is known.

Preferably, the glycosidases are selected from the following enzymes for which a pro-desquamatizing effect has been demonstrated by means of in vitro studies: N-glycanase, cellulases, β-glucosidase, β-galactosidase, N-acetyl-glucosaminidase and/or N-acetyl-galactosaminidase. In a preferred embodiment of the invention, a combination of three glycosidases, β-galactosidase, N-acetyl-glucosaminidase and N-acetyl-galactosaminidase is included in the composition.

The glycosidases used in the compositions of the invention can be purified from extracts of proteins synthesized by cells of the stratum corneum, or they can be glycosidases synthesized naturally by microorganisms. They can also be recombinant glycosidases produced in a heterologous system. Examples of glycosidases that can be used are those sold by CloneZyme™ or by Boehringer.

In general, a composition of the invention comprises 0.001% to 15% by weight of glycosidases, preferably 0.01% to 10%, and more preferably 0.05% to 5% by weight with respect to the total composition weight.

The invention results from the observation that an increase in the glycosidase activity in the stratum corneum is sufficient to encourage desquamation. As a result, the invention also pertains to cosmetic compositions comprising a combination of at least one glycosidase encouraging desquamation with a product that can stimulate glycosidase activity. A product that can stimulate glycosidase activity is a product that can increase the rate of the enzymatic reaction measured, for example, by an increase in the quantity of substrates digested per unit time when said product is added to the reaction medium. An example of a product that is capable of stimulating β-glucosidase activity is 1-O-methyl-β-D-glucopyranoside. Said activators represent between 0.01% and 10% of the total composition weight, preferably between 0.1% and 1% of the total composition weight.

In the compositions for use in the invention as defined above, the glycosidases can be combined with other known active ingredients with desquamating properties, such as hydroxyacids, α- or β-keto-acids, retinoids, certain sulphonic acids or certain carbohydrates such as those defined in L'Oreal's patent application “Use of carbohydrates to encourage skin desquamation”, WO-A-97/12597). Such a combination can reduce the active concentration of said active ingredients due to additive effects. Thus, a less irritating and less toxic composition can be obtained, along with a composition that is more effecfive than those of the prior art using only these active ingredients.

Examples of hydroxyacids are α-hydroxyacids and β-hydroxyacids, which can be saturated or unsaturated, linear, branched or cylic. The hydrogen atoms of the carbonaceous chain can also be substituted with halogens, halogen-containing radicals, or alkyl, acyl, acyloxy, alkoxycarbonyl or alkoxy radicals containing 2 to 18 carbon atoms.

In particular, said hydroxyacids can be the following: glycolic, lactic, malic, tartaric, citric and fruit acids in general, 2-hydroxy alkanoic, mandelic, salicylic acid and their alkylated or acylated derivatives such as n-octanoyl-5-salicylic acid, n-dodecanoyl-5-salicylic acid, n-decanoyl-5-salicylic acid, n-octyl-5-salicylic acid, n-heptyloxy-5- or 4-salicylic acid, 2-hydroxy-3-methylbenzoic acid or their alkoxylated derivatives such as 2-hydroxy-3-methoxybenzoic acid.

In particular, the retinoids can be retinoic acid (all-trans or 13-cis) and its derivatives, retinol (vitamin A) and its esters such as retinol palmitate, retinol acetate and retinol propionate as well as their salts, or retinal.

By way of example, the hydroxyacids, keto-acids and retinoids can be introduced into the compositions of the invention in a quantity representing 0.01% to 5% of the total composition weight, preferably 0.1% to 3%.

The composition used in the invention contains a cosmetically or dermatologically acceptable medium, i.e., a medium that is compatible with the skin, nails, mucous membranes, tissues and hair. In a preferred embodiment of the invention, the pH of the composition allows optimum activity of the glycosidases used, and is preferably close to that of the skin, in the range 4 to 7. The composition comprising one or more glycosidases is preferably applied topically to the face, neck, hair, mucous membranes and the nails or any other cutaneous zone of the body.

A cosmetic composition of the invention is preferably in a suitable form for topical administration and can contain enzyme type active ingredients which are unstable in aqueous media and for topical application. They are usually in the form of hydroalcoholic or oily solutions, lotion or serum type dispersions, anhydrous or oily gels, milk type emulsions with a liquid or semi-liquid consistency obtained by dispersing an oily phase in an aqueous phase (O/W) or vice versa (W/O), suspensions or emulsions with a soft, semi-solid or solid consistency of the cream, gel or micro-emulsion type, or as micro-capsules, micro-particles, or ionic and/or non ionic type vesicular dispersions. Said compositions are prepared using the usual methods. A preferred form that is specially adapted for compositions comprising active enzymes is a W/O/W type as described in L'Oreal's patent application EP-A-0 779 071.

The compositions of the invention can also be used for the hair in the form of alcoholic or hydroalcholic solutions, or in the form of creams, gels, emulsions or foams.

The quantities of the different constituents of the compositions used in accordance with the invention are those that are routinely used in the fields under consideration.

Said compositions constitute creams for protection, treatment or care of the face, hands or body, milks for protecting or caring for the body, lotions, gels or foams for care of the skin and mucous membranes, or for cleaning the skin.

The compositions can also consist of solid preparations constituting soaps or cleaning bars.

In known manner, the composition of the invention can also contain adjuvants that are normal in the cosmetic and dermatological fields, such as hydrophilic or lipophilic gelling agents, hydrophilic or lipophilic active ingredients, preservatives, antioxidants, solvents, fragrances, fillers and colorants. The quantities of said adjuvants are those that are conventionally used in the fields under consideration, for example 0.01% to 20% of the total composition weight. Clearly, the skilled person will be careful to select any additives and/or their quantities so that the advantageous intrinsic properties of the composition of the invention, in particular the enzymatic activities of the glycosidases, are not or are not substantially altered by the envisaged additives.

Oils that can be used in the invention that can be cited are mineral oils (Vaseline oil), vegetable oils (shea oil, sweet almond oil), animal oils, synthesized oils, silicone oils (cyclomethicone), and fluorinated oils (perfluoropolyethers). It is also possible to use fatty alcohols, fatty acids (stearic acid) or waxes (paraffin, carnauba, beeswax) as the oily materials.

Emulsifying agents that can be used in the invention that can be cited are polysorbate 60 and sorbitan stearate sold by ICI under the respective trade names of Tween 60 and Span 60. Co-emulsifying agents can be added, such as PPG-3 myristyl ether sold by Witco as Emcol 249-3K.

Solvents that can be used in the invention that can be cited are lower alcohols, in particular ethanol and isopropanol, and propylene glycol.

Hydrophilic gelling agents that can be cited are carboxyvinyl polymers (carbomers), acrylic copolymers such as acrylate/alkylacrylate copolymers, polyacrylamides, polysaccharides such as hydroxypropylcellulose, natural gums (xanthan), and clays; lipophilic gelling agents that can be cited are modified clays such as bentonites, metallic sols of fatty acids such as aluminium stearates, hydrophobic silica, polyethylenes and ethylcellulose.

Hydrophilic active ingredients that can be used include proteins or protein hydrolysates, amino acids, polyols, urea, allantoin, sugars and sugar derivatives, hydrosoluble vitamins, starch, or bacterial or vegetable extracts, in particular aloe vera.

Lipophilic active ingredients that can be used include tocopherol (vitamin E) and its derivatives, essential fatty acids, ceramides and essential oils.

In order to combat photoaging effectively, it is also possible to add to the composition of the invention one or more complementary sunscreens that are active in the UVA and/or UVB, which may be hydrophilic or hydrophobic, optionally including a sulphonic function.

The sunscreen is preferably selected from organic and/or mineral sunscreens.

Organic sunscreens that can be cited are cinnamic derivatives, salicylic derivatives, canphor derivatives, triazine derivatives, benzophenone derivatives, dibenzoylmethane derivatives, β, β-diphenylacrylate derivatives, p-aminobenzoic acid derivatives, polymeric sunscreens and silicone sunscreens described in patent application WO-A-93/04665, or organic sunscreens described in patent application EP-A-0 487 404.

Mineral sunscreens that can be cited are pigments, or preferably nanopigments (mean primary particle size: generally in the range 5 nm to 10 nm, preferably in the range 10 nm to 50 nm) of coated or uncoated metal oxides, such as nanopigments of titanium oxide (amorphous or crystalline in the form of rutile and/or anatase), iron oxide, zinc oxide, zirconium oxide or cerium oxide, which are all well known photoprotective agents acting by physically blocking (reflection and/or diffusion) UV radiation. Alumina and/or aluminium stearate are conventional coating agents. Such coated or uncoated metal oxide nanopigments have in particular been described in patent applications EP-A-0 518 772 and EP-A-0 518 773.

Examples of complementary sunscreens that are active in the UV-A and/or UV-B region that can be cited are:

p-aminobenzoic acid;

oxyethylenated (25 mol) p-aminobenzoate;

2-ethylhexyl p-dimethylaminobenzoate;

N-oxypropylenated ethyl p-aminobenzoate;

glycerol p-aminobenzoate;

homomenthyl salicylate;

2-ethylhexyl salicylate;

triethanolamine salicylate;

4-isopropylbenzyl salicylate;

4-tert-butyl-4′-methoxy-dibenzoylmethane (PARSOL 1789 from GIVAUDAN ROURE);

2-ethylhexyl p-methoxycinnamate (PARSOL MCX from GIVAUDAN ROURE);

4-isopropyl-dibenzoylmethane (EUSOLEX 8020 from MERCK); menthyl anthranilate;

2-ethylhexyl-2-cyano-3,3′-diphenylacrylate (UVINUL N539 from BASF);

ethyl-2-cyano-3,3′-diphenylacrylate;

2-phenyl benzimidazole 5-sulphonic acid and its salts;

3-(4′-trimethylammoniun)-benzylidene-bornan-2-one-methylsulphate;

2-hydroxy-4-methoxybenzophenone (UVINUL MS 40 from BASF);

2-hydroxy-4-methoxybenzophenone-5-sulphonate (UVINUL MS 40 from BASF);

2,4-dihydroxybenzophenone (UVINUL 400 from BASF);

2,2′,4,4′-tetrahydroxybenzophenone (UVINUL D 50 from BASF);

2,2′-dihydroxy-4,4′-dimethoxybenzophenone (HELOSORB II from NORQUAY);

2-hydroxy-4-n-octoxybenzophenone;

2-hydroxy-4-methoxy-4′-methylbenzophenone;

α-(2-oxoborn-3-ylidiene)-tolyl-4-sulphonic acid and its salts;

3-(4′-sulpho)benzylidene-bornan-2-one and its salts;

3-(4′-methylbenzylidene)-d,1-camphor;

3-benzylidene-d,1-camphor;

benzene 1,4-di(3-methylidene-10-camphorsulphonic) acid and its salts (MEXORYL SX from CHIMEX);

urocanic acid;

2,4,6-tris-[p-(2′-ethylhexyl-1′-oxycarbonyl)anilino]-1,3,5-triazine;

2-[p-(tertiobutylamido)anilino]-4,6-bis[p-(2′-ethylhexyl-1′-oxycarbonyl)anilino]-1,3,5-triazine;

2,4-bis{[4-2-ethylhexyloxyl]-2-hydroxyphenyl}-6-(4-methoxyphenyl)-1,3,5-triazine;

the polymer of N-(2 and 4)-[2-oxoborn-3-ylidene)methyl)benzyl]-acrylamide;

4,4-bis-benzimidazolyl-phenylene-3,3′,5,5′-tetrasulphonic acid and its salts;

2,2′-methylene-bis-[6-(2H-benzotriazole-2-yl)-4-(1,1,3,3-tetramethylbutyl)phenol];

polyorganosiloxanes with a malonate function.

The invention also concerns a cosmetic treatment method implemented by applying compositions as defined above using the normal technique for using said compositions. Examples are: application of creams, gels, serums, ointments, lotions, milks to the skin, the scalp, the nails and/or the mucous membranes.

The compositions that encourage desquamation are suitable for treating dry skin or combating cutaneous aging.

As a result, the invention also concerns the use of compositions as described above in treating dry skin or for the treatment and/or prevention of cutaneous aging.

The following section shows the results of an in vitro study of the effects of different glycosidases on desquamation. It also shows non-limiting examples of formulations with the compositions of the invention.

### EXPERIMENTAL SECTION

### Key to Figures

FIG. 1 is a histogram illustrating the effect of 5% urea on corneocyte detachment. The values are shown as a percentage of the level of keratin detection with respect to a negative control containing the PBS base buffer (100%).

FIG. 2 illustrates the dose-response effect of N-glycanase on corneocyte detachment compared with urea (by immunoblot).

FIG. 3 is a graph illustrating the effect of N-glycanase in three different concentrations on corneocyte detachment. The values are mean values of 5 independent experiments expressed as a percentage of the level of detection of keratins with respect to a negative control containing the base buffer (100%).

FIG. 4 illustrates the effect of certain glycosidases and certain cellulases sold by CloneZyme on desquamation, by immunoblot.

FIG. 5 is a graph illustrating the effect of cellulase on corneocyte detachment compared with the base buffer. The values are shown as a percentage of the level of detection of keratins with respect to a negative control containing the base buffer (100%).

FIG. 6 is a graph illustrating the effect of a mixture of glycosidases on corneocyte detachment. The values are shown as a percentage of the level of detection of keratins with respect to a negative control containing the base buffer (100%).

### 1. IN VITRO “DESQUAMATION” TEST ADAPTED TO SCREENING MOLECULES AND VALIDATION BY STUDYING DIFFERENT GLYCOSIDASES

### 1.1. Development of an in vitro Desquamation Test Suitable for Molecule Screening

1.1.1. Method

In order to recover samples of the superficial layers of the stratum corneum, the test employed the Blenderm-varnish method carried out on the calves. Varnish was applied directly to the skin then, after 10 minutes, the superficial layers of the stratum corneum were removed with Blenderm type adhesive tape (3M). The filter containing the sample was then fitted to a 96 well Teflon microtitration plate and fixed on a base that sealed the wells from each other.

90 μl of base buffer (PBS, pH 6.5, 0.1% sodium acid, 0.05% Tween 20) containing the enzymes or test products was added per well. Each determination was carried out on 3 or 4 wells distributed differently on the plate after an incubation period in the range 1 hour to 48 hours at a temperature in the range 20° C. to 37° C. 40 μl from each well treated under the same conditions was mixed and filtered through a sieve with a pore size of 0.22 μm to eliminate soluble proteins and retain the released corneocytes. The corneocytes were taken up in 30 μl of base buffer in the presence of DTT and heated for 15 minutes at 80° C. The samples were then centrifuged to eliminate the insoluble material and analyzed by SDS-PAGE and/or Western Blot using anti-keratin K10 monoclonal antibodies (suprabasal keratins). The keratins were detected after staining the proteins with silver nitrate (to an apparent molecular weight of about 55-65 kDa). The use of an anti-K10 monoclonal antibody could confirm the presence of dissolved suprabasal keratins.

1.1.2. Results

An analysis of the fractions passed through the sieve with pore size 0.22 μm (containing the soluble proteins) contained no proteins mainly represented by 55-65 kDa corresponding to the keratin migration zone. In contrast, an analysis of the fraction retained by the filter allowed the keratins to be identified. This confirmed that keratin analysis by immunoblot correctly reflects the presence of corneocytes, the corneocyte keratins being bound to the insoluble material. Quantification of the detected keratins can thus produce an estimation of the quantity of corneocytes released during incubation and thus an evaluation of the pro-desquamating effect of a product.

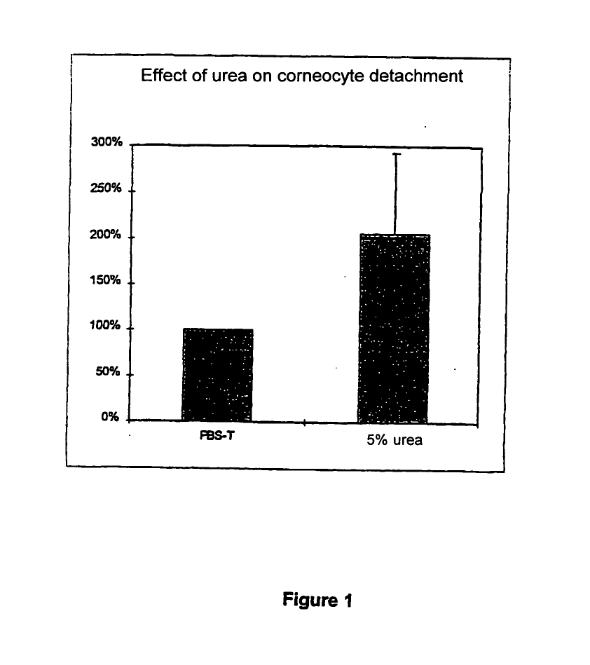

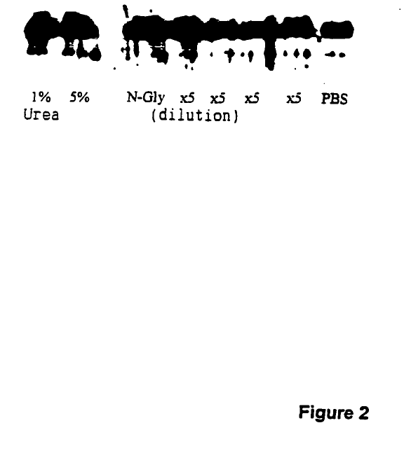

Urea is a component that is known to have a positive effect on desquamation. In order to validate the test, this molecule was used as a positive control in concentrations of 1% and 5% (in comparison with the negative control constituted by the buffer base alone). The results are shown in FIGS. 1 and 2. The mean data for tests carried out in 4 independent experiments clearly show an effect of urea of 5% (200%±100% of keratin measured compared with 100% measured without urea).

These results validate the use of this test for screening molecules that may encourage desquamation.

### 1.2. Studies of the Effect of Different Glycosidases on Desquamation

1.2.1. Study of N-glycanase (Endo-F from Flavobacterium Meningosepticum) on Desquamation

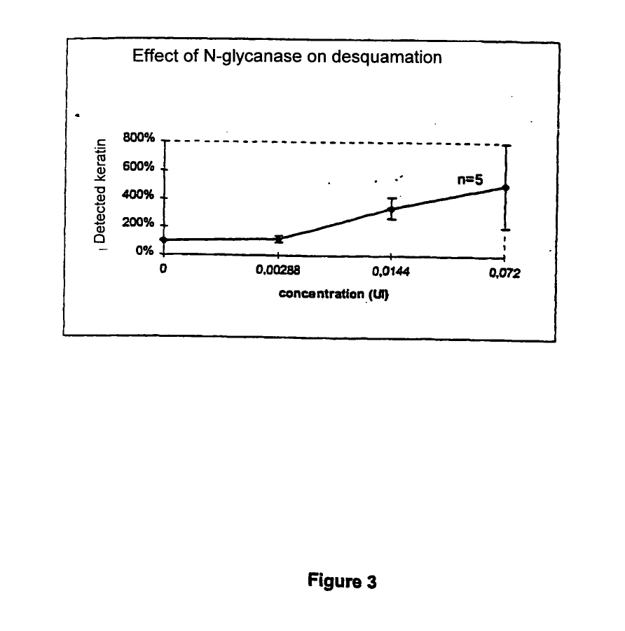

N-glycanase (endo F) can eliminate all N-bound chains carried by glycoproteins. The N-glycanase used was that sold by Boehringer. The results shown in FIGS. 2 and 3 clearly show an effect of N-glycanase on corneocyte detachment.

1.2.2. Study of Thermostable Recombinant Glycosidases on Desquamation

Different glycosidases sold by CloneZyme™ were tested for their capacity to encourage desquamation. Table 1 below shows the substrate specificities for the different glycosidases tested.

TABLE 1

| | GLY- | GLY- | GLY- | GLY- | GLY- | GLY- | GLY- | GLY- | GLY- | GLY- |
|---|---|---|---|---|---|---|---|---|---|---|
| | 01 | 02 | 03 | 04 | 05 | 06 | 07 | 08 | 09 | 10 |
| β-D-cellobiose | − | ++ | ε | ε | | + | + | ε | ε | − |
| β-D-galactose | − | ++ | ε | + | + | ε | ε | + | − | − |
| α-D-glucose | − | − | − | − | − | − | − | − | − | − |
| β-D-glucose | ++ | ++ | ++ | ++ | ++ | ++ | ++ | ++ | ++ | − |
| β-N-acetyl- | − | − | − | − | − | − | − | − | − | − |
| D-glucosaminide | | | | | | | | | | |
| β-D-fucose | − | ++ | ++ | ++ | ++ | ++ | ++ | + | ++ | − |
| β-L-fucose | − | − | − | − | − | − | − | − | − | − |
| β-D-glucuronide | − | − | − | − | − | − | ε | − | − | − |
| α-D-galactose | − | − | − | − | − | − | − | − | − | + |
| β-D-mannose | − | ε | − | − | − | + | + | − | + | − |
| α-D-mannose | − | − | − | − | − | − | − | − | − | − |
| β-D-xylose | ε | + | − | ε | ε | ε | ε | ε | ε | − |
| α-L- | − | − | − | − | − | − | − | − | − | − |
| arabinofuranoside | | | | | | | | | | |
| α-L- | − | + | ε | ε | ε | ε | ε | ε | ε | − |
| arabinopyranoside | | | | | | | | | | |
| β-D-lactose | − | + | ε | − | − | + | ε | − | − | − |
| α-L-rhamnose | − | − | − | − | − | − | − | − | − | − |
| α-D-N- | − | − | − | − | − | − | − | − | − | − |
| acetylneuramide | | | | | | | | | | |
| β-D-N- | − | − | − | − | − | − | − | − | − | − |
| acetylchitobioside | | | | | | | | | | |
| α-L-fucose | − | − | − | − | − | − | − | − | − | − |

(−: no activity; ε, +, ++: specific activity)

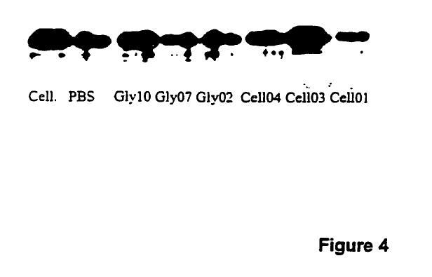

One advantage of these glycosidases is their relatively ready availability and their stability to temperature variations. As a result, they are capable of being used in preparing compositions that are suitable for topical administration. By identifying keratins after staining SDS-PAGE gels with silver nitrate and immunodetection, the results shown in FIG. 4 show that these glycosidases encourage desquamation. The specificity of these glycosidases towards their substrates is more or less close. The results show optimum efficacy with the glycosidase Gly 10 with, in order of efficacy, Gly10>Gly07>Gly02.

1.2.3. Study of the Effect of Glycosidases on Desquamation

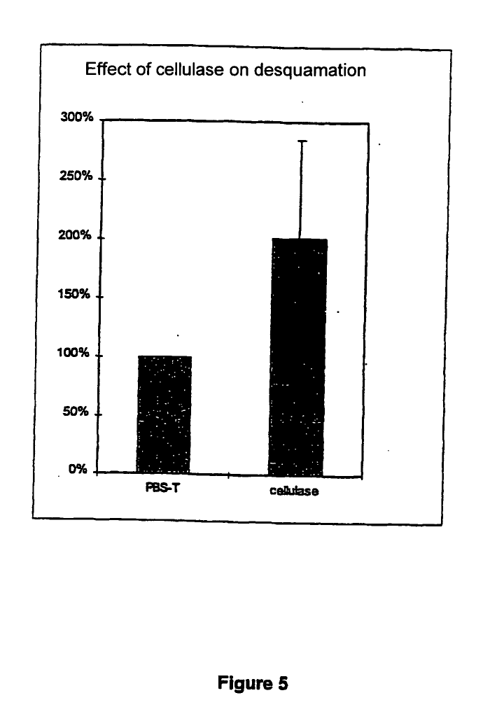

Cellulases cleave the β1-4 bonds located between two internal glucoses (endoglycosidases). The presence of cellulose or similar structures in the human epidermis has not been described. However, it is possible that saccharide structures in the stratum corneum could constitute cellulase targets. The effect of cellulase sold by Boehringer has been tested for its capacity to encourage desquamation. The results shown in FIG. 5 show surprisingly a substantial effect (similar to urea) of cellulase on desquamation. Similar results were obtained with several recombinant cellulases sold by CloneZyme™, with an order of efficiency of cell03>cell04, cell01 (FIG. 4).

Table 2 shows the substrate specificity of different test cellulases as regards glycosides.

TABLE 2

| | CELLULASES (CLONEZYME) | | | | | | |
|---|---|---|---|---|---|---|---|
| | CEL- | CEL- | CEL- | CEL- | CEL- | CEL- | CEL- |
| Substrat | 001 | 002 | 003 | 004 | 005 | 006 | 007 |
| β-D-cellobiose | ++ | ++ | − | ++ | − | − | − |
| β-D-galactose | + | − | − | − | − | − | − |
| α-D-glucose | − | − | − | − | − | − | − |
| β-D-glucose | ++ | − | − | − | − | − | − |
| β-N-acetyl- | − | − | − | − | − | − | − |
| D-glucosaminide | | | | | | | |
| β-D-fucose | ++ | − | − | − | − | − | − |
| β-L-fucose | − | − | − | − | − | − | − |
| β-D-glucuronide | − | − | − | − | − | − | − |
| α-D-galactose | − | − | − | + | − | ++ | − |
| β-D-mannose | ++ | − | ++ | ++ | − | − | − |
| α-D-mannose | − | − | − | − | − | − | − |
| β-D-xylose | + | − | − | − | − | − | − |
| α-L-arabinofuranoside | − | − | − | − | − | − | − |
| α-L-arabinopyranoside | + | − | − | − | − | − | − |
| β-D-lactose | ++ | ++ | − | ++ | − | − | − |
| α-L-rhamnose | − | − | − | − | − | − | − |
| α-D-N- | − | − | − | − | − | − | − |
| acetylneuramide | | | | | | | |
| β-D-N- | − | − | − | − | − | − | − |
| acetylchitobioside | | | | | | | |
| α-L-fucose | − | − | − | − | − | − | − |

(+: 1-10% relative activity; ++ 10-100% relative activity

1.2.4. Effects of Different Exo-glycosidases and O-glycanases

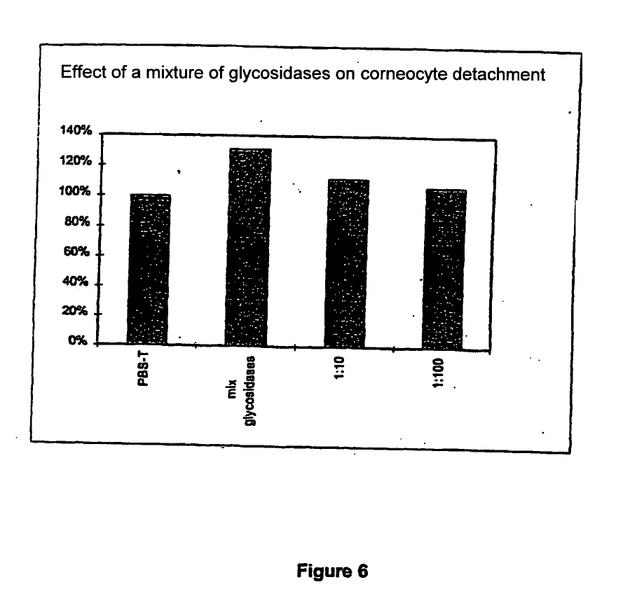

Certain exo-glycosidases were tested for their effect on desquamation. In particular, 40 U of β-galactosidase, of α-fucosidase, 1 U of N-acetyl-glucosaminidase, 0.5 U of α-galactosidase, 0.3 U of α-mannosidase, 0.2 U of β-mannosidase, 50 U of glucuronidase, 5 U of β-glucosidase and 5 U of α-glucosidase were tested in vitro. Taken separately, exoglycosidases only very slightly affect desquamation. The combination that showed a significant effect on desquamation was a mixture of β-galactosidase, N-acetyl-glucosaminidase and N-acetyl-galactosaminidase. FIG. 6 shows the effect of this combination of enzymes on corneocyte detachment.

O-glycanase can specifically cleave O-bonded chains. O-glycanase was tested for its capacity to encourage desquamation. The results obtained in in vitro tests showed that O-glycanase not, or only very slightly, to affect desquamation (10% increase observed). Further, the effect of the simultaneous use of O-glycanase and broad spectrum sialidase (Arthrobacter ureafaciens) or O-glycanase+N-glycanase and sialidase was no better than that observed with N-glycanase alone.

### 2. EXAMPLES OF FORMULATIONS FOR COMPOSITIONS OF THE INVENTION

### Composition 1: Face Milk

| Vaseline oil | 7.0 | g |
|---|---|---|
| N-glycanase (endo-F from Flavobacterium | 0.1 | g |
| meningosepticum) | | |
| Glyceryl monostearate, polyethylene glycol stearate (100 | 3.0 | g |
| OE) | | |
| Carboxyvinyl polymer | 0.4 | g |
| Stearyl alcohol | 0.7 | g |
| Soya proteins | 3.0 | g |
| NaOH | 0.4 | g |
| Preservative | qs | |
| Water | qsp 100 | g |

This composition was in the form of a face milk with good cosmetic properties and was mild and comfortable in use.

The pH of the composition was about 5.5.

### Composition 2: Lotion

| β-glucosidase | 0.5 | g |
|---|---|---|
| 2-ethylhexyl palmitate | 10.0 | g |
| Cyclopentadimethylsiloxane | 20.0 | g |
| Butylene glycol | 5.0 | g |
| Preservative | qs | |
| Water | qsp 100 | g |

This lotion, which contained no surfactants, encouraged skin desquamation.

### Composition 3: Milk

| Octyl palmitate | 35.0 | g |
|---|---|---|
| Glycerin | 2.0 | g |
| N-acetyl-glucosaminidase | 0.8 | g |
| C10-C30 acrylate/alkylacrylate cross-linked polymer | 0.1 | g |
| Triethanolamine | 0.1 | g |
| Wheat amino acids | 1.0 | g |
| Preservative | qs | |
| Water | qsp 100 | g |

The milk obtained, which contained no surfactants, had good cosmetic properties.

### Composition 4: Face Gel

| Glycerin | 10.0 | g |
|---|---|---|
| N-acetyl-galactosidase | 1.0 | g |
| Disodium cocoamphodiacetate | 1.0 | g |
| Preservative | qs | |
| Water | qsp 100 | g |

The gel obtained had good cosmetic properties.

### Compositi n 5: Water Cleansing Gel

| Butylene glycol | 7.0 | g |
|---|---|---|
| Sodium lauroyl sarcosinate | 4.0 | g |
| β-glucosidase | 1.0 | g |
| Triethanolamine | 0.8 | g |
| Carbomer | 0.5 | g |
| Preservative | qs | |
| Water | qsp 100 | g |

The gel obtained had good cosmetic properties.

1. A composition, characterized in that it comprises at least one glycosidase encouraging desquamation as the active principle, said composition containing no proteases.

2. A composition according to claim 1, characterized in that the glycosidase or glycosidases is/are selected by an in vitro desquamation test comprising the following steps:
a) recovering samples of stratum corneum from an individual;
b) fixing the sample onto a support;
c) bringing the sample fixed on the support into contact with a buffered solution containing the glycosidase to be tested;
d) recovering insoluble material released into the buffered solution;
e) assaying the keratins present in the insoluble material and quantifying them with respect to a test control without glycosidase.

3. A composition according to claim 1 or claim 2, characterized in that the glycosidase or glycosidases is/are selected from the following enzymes: N-glycanase, cellulases, β-glucosidase, β-galactosidase, N-acetyl-glucosaminidase and/or N-acetyl-galactosaminidase.

4. A composition according to claim 3, characterized in that it comprises a combination of β-galactosidase, N-acetyl-glucosaminidase and N-acetyl-galactosaminidase.

5. A composition according to any one of claims 1 to 4, characterized in that it further comprises a product that can stimulate glycosidase activity.

6. A composition according to any one of claims 1 to 5, characterized in that the glycosidases encouraging desquamation represent in the range 0.001% to 15% of the total composition weight, preferably in the range 0.01% to 10% and more preferably in the range 0.05% to 5%.

7. A composition according to claim 5, characterized in that the product that can stimulate glycosidase activity represents 0.01% to 10% of the total composition weight, preferably 0.1% to 1%.

8. A composition according to any one of claims 1 to 7, characterized in that at least one glycosidase encouraging desquamation is a recombinant glycosidase.

9. A composition according to one of claims 1 to 8, characterized in that at least one glycosidase is a glycosidase isolated from the stratum corneum.

10. A composition according to one of claims 1 to 9, characterized in that it further comprises at least one other active ingredient having properties that encourage desquamation.

11. A composition according to one of claims 1 to 10, characterized in that it is suitable for topical administration.

12. Use of a composition according to any one of claims 1 to 11, for treating dry skin or for treating and/or preventing cutaneous aging.

13. A method for the cosmetic treatment of the skin to encourage desquamation of the skin, consisting of applying a composition according to claim 11 to the skin, scalp, nails and/or mucous membranes.

14. A cosmetic method for treating dry skin, for preventing and/or treating cutaneous aging, consisting of applying a composition according to claim 11 to the skin.
